(19) United States
(12) Patent Application Publication
(10) Pub. No.: US 2022/0371604 A1
(43) Pub. Date: Nov. 24, 2022

(54) SYSTEMS AND METHODS TO PROTECT HEALTH OF OCCUPANTS OF A VEHICLE

(71) Applicant: Ford Global Technologies, LLC, Dearborn, MI (US)

(72) Inventors: Bhagyashri Katti, Novi, MI (US); Nithya Somanath, Farmington Hills, MI (US); Fling Finn Tseng, Ann Arbor, MI (US); Nikhil Jamdade, Dearborn, MI (US); Shiqi Qiu, Canton, MI (US)

(73) Assignee: Ford Global Technologies, LLC, Dearborn, MI (US)

(21) Appl. No.: 17/323,768

(22) Filed: May 18, 2021

(51) Int. Cl. B60W 50/00; B60W 40/08

(52) U.S. Cl. CPC B60W 50/0098; B60W 40/08; B60W 2540/215

(57) ABSTRACT

This disclosure is generally directed to systems and methods for protecting the health of occupants of a vehicle. In an example method, a sensor in a vehicle sends sensor data to a computer, based on detecting a symptomatic feature that indicates a health status of a first occupant of the vehicle. In one case, the symptomatic feature may be an elevated body temperature that may be symptomatic of a fever. In another case, the detected symptomatic feature may be a sound emitted by the first occupant such as, for example, a cough, a sneeze, or a wheeze. The computer evaluates the sensor data and identifies a health risk posed by the first occupant to a second occupant of the vehicle. The computer may address the health risk, for example, by modifying an air quality inside the vehicle or by instructing the occupants of the vehicle to don masks.

### BACKGROUND

A ride share vehicle may be occupied at various time by various people who may contribute to the spread of a virus. In some cases, a first individual who uses a ride share vehicle may suffer from a health issue such as, for example, a cold, a fever, or a communicable disease that can be transmitted to a second individual who may be sharing a ride with the first occupant in the ride share vehicle or, in some cases, may enter the ride share vehicle after the first individual has exited the ride share vehicle.

It is therefore desirable to address the issue described above and provide a solution for protecting the health of occupants of a vehicle.

### BRIEF DESCRIPTION OF THE DRAWINGS

A detailed description is set forth below with reference to the accompanying drawings. The use of the same reference numerals may indicate similar or identical items. Various embodiments may utilize elements and/or components other than those illustrated in the drawings, and some elements and/or components may not be present in various embodiments. Elements and/or components in the figures are not necessarily drawn to scale. Throughout this disclosure, depending on the context, singular and plural terminology may be used interchangeably.

FIG. 1 shows an example system that offers health protection to occupants of a vehicle in accordance with an embodiment of the disclosure.

FIG. 2 shows some example components that may be included in a vehicle in accordance with an embodiment of the disclosure.

FIG. 3 shows some example components that may be included in a personal communication device of an occupant of a vehicle in accordance with an embodiment of the disclosure.

FIG. 4 shows a flowchart of an example procedure to analyze a sound produced by an occupant of a vehicle for identifying a health status of the occupant.

FIG. 5 shows a graphical representation of various types of example sound patterns that may be analyzed in order to determine a health status of an occupant of a vehicle.

### DETAILED DESCRIPTION

### Overview

In terms of a general overview, certain embodiments described in this disclosure are directed to systems and methods related to protecting the health of occupants of a vehicle. In an example method, a sensor in a vehicle detects a symptomatic feature that indicates a health status of a first occupant of a vehicle. The sensor sends sensor data to a computer based on the detected symptomatic feature. In one case, the detected symptomatic feature may be an elevated body temperature of the first occupant that may be symptomatic of a fever. In another case, the detected symptomatic feature may be a sound emitted by the first occupant such as, for example, a cough, a sneeze, or a wheeze. The computer evaluates the sensor data and identifies a health risk posed by the first occupant to a second occupant of the vehicle. The computer may address the health risk posed by the first occupant to a second occupant of the vehicle in various ways such as, for example, by modifying an air quality inside the vehicle and/or by instructing the occupants of the vehicle to don masks.

### Illustrative Embodiments

The disclosure will be described more fully hereinafter with reference to the accompanying drawings, in which example embodiments of the disclosure are shown. This disclosure may, however, be embodied in many different forms and should not be construed as limited to the example embodiments set forth herein. It will be apparent to persons skilled in the relevant art that various changes in form and detail can be made to various embodiments without departing from the spirit and scope of the present disclosure. Thus, the breadth and scope of the present disclosure should not be limited by any of the above-described example embodiments but should be defined only in accordance with the following claims and their equivalents. The description below has been presented for the purposes of illustration and is not intended to be exhaustive or to be limited to the precise form disclosed. It should be understood that alternate implementations may be used in any combination desired to form additional hybrid implementations of the present disclosure. For example, any of the functionality described with respect to a particular device or component may be performed by another device or component. Furthermore, while specific device characteristics have been described, embodiments of the disclosure may relate to numerous other device characteristics. Further, although embodiments have been described in language specific to structural features and/or methodological acts, it is to be understood that the disclosure is not necessarily limited to the specific features or acts described. Rather, the specific features and acts are disclosed as illustrative forms of implementing the embodiments.

Certain words and phrases are used herein solely for convenience and such words and terms should be interpreted as referring to various objects and actions that are generally understood in various forms and equivalencies by persons of ordinary skill in the art. For example, it must be understood that words such as “sanitize” and “sanitizing” as used herein are intended to encompass various other words such as “clean,” “cleaning,” “disinfect,” “disinfecting,” “wash,” and “washing.” In general, and in accordance with disclosure, these words pertain to removal of undesirable pollutants such as, for example, viruses, bacteria, dirt, and dust that may be present in a vehicle. Words such as “sensor” and “detector” may be used interchangeably in this disclosure and must be understood as being equivalent where applicable. The word “occupant” as used herein refers to any occupant of a vehicle such as, for example, a driver of a vehicle or a passenger of a vehicle. The phrase “symptomatic feature” as used herein refers to any state of an object, condition of an object, or occurrence of an event, that pertains to a communicable disease. The word “disease” as used herein encompasses all types of undesirable health conditions including fever, pain, and illness. It should also be understood that the word “example” as used herein is intended to be non-exclusionary and non-limiting in nature.

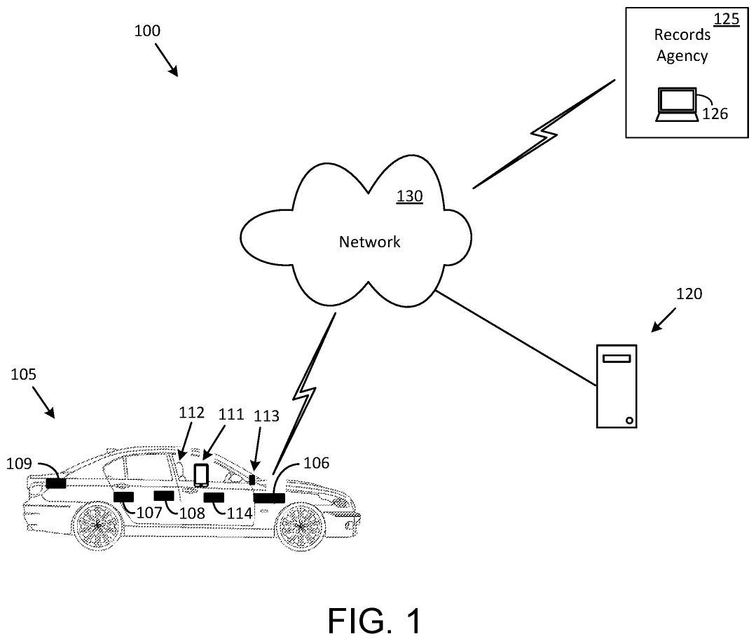

FIG. 1 shows an example system 100 that offers health protection to occupants of a vehicle 105 in accordance with an embodiment of the disclosure. The vehicle 105 may be any of various types of vehicles such as, for example, a car, a van, a sports utility vehicle, a truck, an electric vehicle, a gasoline vehicle, a hybrid vehicle, a driver-operated vehicle or an autonomous vehicle. The example illustration of the vehicle 105 shows a driver 112 who is one example occupant of the vehicle 105, which can be a ride-share vehicle in some cases. In other scenarios, the vehicle 105 can be an autonomous vehicle, and all the occupants of the autonomous vehicle may be passengers.

The vehicle 105 may include components such as a vehicle computer 106, a pollutant detection system 107, a vehicle sanitizer system 108, a wireless communication system 109, an infotainment system 113, and sanitizing hardware 114. The components, which are symbolically depicted as black boxes in FIG. 1, may be installed at various locations on the vehicle 105, such as, for example, an engine compartment, a glove compartment, a trunk, a console inside the cabin area, or an exterior portion of the vehicle 105. In some cases, some of the components, such as, for example, sanitizing hardware 114, may be provided outside the vehicle 105.

The vehicle computer 106 may perform various functions such as, for example, controlling engine operations (fuel injection, speed control, emissions control, braking, etc.), managing climate controls (air conditioning, heating etc.), activating airbags, and issuing warnings (check engine light, bulb failure, low tire pressure, vehicle in blind spot, etc.). In some cases, the vehicle computer 106 may include more than one computer such as, for example, a first computer that controls engine operations and a second computer that operates the infotainment system 113.

The vehicle sanitizer system 108 is configured to execute various operations in accordance with the disclosure. Such operations may include cooperating with the wireless communication system 109 to wirelessly communicate with various systems and devices via a network 130. The network 130 may include any one, or a combination of networks, such as a local area network (LAN), a wide area network (WAN), a telephone network, a cellular network, a cable network, a wireless network, and/or private/public networks such as the Internet. For example, the network 130 may support communication technologies such as Bluetooth®, cellular, near-field communication (NFC), Wi-Fi, Wi-Fi direct, machine-to-machine communication, and/or man-to-machine communication. At least one portion of the network 130 includes a wireless communication link that allows the vehicle sanitizer system 108 to communicate via the wireless communication system 109 with a server computer 120 and/or a computer 126 that is located in a records agency 125.

The pollutant detection system 107 may be implemented in any of various ways. In an example implementation, the pollutant detection system 107 can include multiple cameras that are mounted at various locations in a cabin area of the vehicle 105. In an example implementation, the cameras may be arranged to capture images of one or more occupants present in the cabin area of the vehicle 105 (clothes, face, body, limbs, etc.). In another example implementation, the cameras may be arranged to capture images of various objects located in the cabin area such as, for example, the seats, the dashboard, the steering wheel, and other fixtures. The images are conveyed to the vehicle sanitizer system 108, which may evaluate the images for identifying a health risk that may be posed by a first occupant of the vehicle 105 to another occupant of the vehicle 105 and/or to detect various undesirable pollutants that may be present on one or more of the various objects present in the cabin area of the vehicle 105.

The pollutant detection system 107 may further include one or more sensors mounted at various locations in a cabin area of the vehicle 105 and/or on an exterior portion of the vehicle 105. The sensors may be selected and configured for capturing data pertaining to various types of pollutants that may be present in the air, on objects, and/or on individuals in the cabin area of the vehicle 105.

In an example implementation, a first sensor may be a thermal sensor (or an infrared imager) that is arranged in a cabin area of the vehicle 105 to capture a body temperature measurement of an occupant in the vehicle 105. The body temperature is an example of a symptomatic feature that indicates a health status of the occupant in the vehicle 105. An elevated body temperature can indicate a fever or an illness that may be communicable and poses a health risk to other occupants of the vehicle 105. The body temperature measured by the thermal sensor may be propagated to a computer such as included in the vehicle sanitizer system 108 or the server computer 120. The computer evaluates the measured body temperature such as, for example, by comparing the measured body temperature to a nominal normal body temperature (98.4° F.). If the measured body temperature is an elevated body temperature (greater than the nominal normal body temperature) the vehicle sanitizer system 108 may execute various actions to protect the occupants of the vehicle 105. Some example actions may involve modifying an air flow inside the cabin area of the vehicle 105 and/or providing instructions to the occupants of the vehicle 105 for minimizing risk. The instructions may be provided through the infotainment system 113 of the vehicle 105 and may include, for example, an instruction to move seating positions so as to maintain a person-to-person separation distance, an instruction to apply a sanitizer, and/or an instruction to don a mask. In some cases, the sanitizer and/or mask(s) may be made available in the cabin area of the vehicle 105.

In another example implementation, an audio sensor may be arranged in a cabin area of the vehicle 105 for capturing sounds produced by one or more occupants of the vehicle 105. The audio sensor, which can be a microphone, may propagate a sound sample produced by an occupant of the vehicle 105 to a computer that is a part of the vehicle sanitizer system 108 and/or the server computer 120. The computer may analyze the sound samples to detect sounds such as, for example, a cough, a sneeze, or a wheeze. Such sounds constitute another example of a symptomatic feature that indicates a health status of the occupant in the vehicle 105. In an example analysis procedure, sound samples accumulated over a period of time may be analyzed by using machine learning techniques and/or a pattern recognition algorithm.

In one scenario, the computer may identify a health issue associated with occupant of the vehicle 105 based on analyzing sound samples produced by the occupant. The vehicle sanitizer system 108 may execute various actions to protect the occupants of the vehicle 105 such as, for example, modifying an air flow inside the cabin area of the vehicle 105 to direct air away from the occupant and/or instructing the occupants of the vehicle 105 to carry out certain actions to minimize risk. The instructions may be provided through the infotainment system 113 of the vehicle 105 and may include, for example, an instruction to move seating positions so as to maintain a person-to-person separation distance, an instruction to apply a sanitizer, and/or an instruction to don a mask. In some instances, the system may know a number of diseases and symptoms of the diseases. As noted above, the system may observe one or more occupants, and if there are any identifiable symptoms, the system may classify if the occupant has any of the diseases.

In another example implementation, an air quality sensor such as, for example, a particulate matter sensor, may be employed to measure an air quality of air outside the vehicle 105. The air quality sensor may be mounted on an exterior portion of the vehicle 105 such as, for example, near an air vent opening in a front portion of the vehicle, a trunk lid of the vehicle 105, or a roof of the vehicle 105. In an example scenario, the air quality outside the vehicle may be poor and it would be undesirable to allow this poor-quality air to enter the cabin area of the vehicle 105. An air quality parameter measured by the air quality sensor may be propagated to a computer such as included in the vehicle sanitizer system 108, or the server computer 120. The computer evaluates the measured air quality parameter such as, for example, by comparing the measured air quality parameter to a threshold air quality standard. If the measured air quality parameter is below the threshold air quality standard, the vehicle sanitizer system 108 may execute various actions to protect the occupants of the vehicle 105 such as, for example, shutting the air intake vent, re-circulating the air inside the cabin, and/or activating high-efficiency particulate air (HEPA) filtering, so as to prevent airborne pollutants (allergens, bacteria, etc.) from entering the vehicle 105.

The air quality measurements may be carried out by the air quality sensor under control of the vehicle sanitizer system 108, at various times and/or at various locations along a travel route of the vehicle 105. For example, air quality measurements may be carried out more frequently during certain times of the year when the pollution level in the air is expected to be high and/or may be carried out at certain high pollution areas along a travel route of the vehicle 105 (high traffic density areas, industrial areas, smog areas, etc.). Air quality maps may be used for this purpose.

In another example implementation, a bacteria detector and/or a chemical detector may be selected and configured to detect various pollutants and undesirable elements in the cabin area of the vehicle 105. More particularly, the bacteria detector may be configured to detect bacteria and/or other biological pollutants and the chemical detector may be configured to detect chemical pollutants in the cabin area of the vehicle 105. Data provided by the sensors to the vehicle sanitizer system 108 may be evaluated by the vehicle sanitizer system 108 for detecting a presence, and/or level, of such pollutants if present in the cabin area of the vehicle 105 and execute preventive and/or remedial actions (modifying air flow, instructing occupants, and/or carrying out disinfecting procedures).

In some applications, the vehicle sanitizer system 108 may perform sanitization procedures when there are no occupants present in the vehicle. A scheduling routine may be applied in order to determine a time for carrying out a sanitization procedure such as, for example, when the vehicle 105 is a ride share vehicle and no rides are scheduled. The sanitization procedure can include one or more operations such as, for example, dispensing an aerosol (antibacterial agent, disinfectant, deodorizer, air freshener, etc.) in the cabin area of the vehicle 105, dispensing a liquid or a gel upon a surface (seat, dashboard etc.), shining ultraviolet (UV) light upon a surface, and/or cleaning a surface (seat, dashboard etc.) with a sanitizing agent (a liquid soap, a disinfectant, a sterilizer, an antiseptic, etc.). In some applications, additional actions may be carried out by human operators, such as, for example, changing out seat covers and other items that may be used for covering objects in the vehicle 105 (knobs, handles, console etc.). These actions may be carried out, for example, during periods after a passenger exits the vehicle 105 and before another enters the vehicle 105.

The sanitizing agents (antibacterial agent, disinfectant, sterilizer, antiseptic, deodorizer, soap, etc.) may be selected on the basis of various factors. In one case, the sanitizing agents may be selected on the basis of their cleaning characteristics and the type of pollutants present in the cabin area (dirt, liquid stains, viruses, bacteria, allergens etc.). In another case, the sanitizing agents may be selected on the basis of a season. A first sanitizing agent may be selected during the spring season, for example, for purposes of removing allergens that may be present in the cabin area during this season. A second sanitizing agent may be selected during the winter season, for example, for purposes of eliminating pathogens (flu virus, COVID-19 virus, bacteria, etc.) that may be more prevalent at this time of the year. In yet another case, the sanitizing agents may be selected on the basis of passenger preference.

In an example application, sensor data and/or image evaluation may be carried out by the vehicle sanitizer system 108 in order to detect a level of pollutants that may be present inside the cabin area of the vehicle 105 before, and/or after, a sanitizing procedure has been executed. A level of cleanliness of the cabin area may be determined by the vehicle sanitizer system 108 by comparing the detected level of pollutants in the cabin area to a threshold level that may be stored in a database. The sanitizing procedure may be terminated when the level of pollutants is below the threshold level.

In another example implementation, the vehicle sanitizer system 108 may wirelessly communicate with the computer 126 in the records agency 125 and/or a personal communication device 111 of an occupant of the vehicle 105. The records agency 125 can be, for example, a medical practice or a public health records office. The personal communication device 111 can be, for example, a smartphone, a tablet computer, a phablet (phone plus tablet computer), or a laptop computer.

The vehicle sanitizer system 108 may wirelessly communicate with the computer 126 in the records agency 125 for obtaining a health history of an occupant of the vehicle 105 and/or for information about a disease. The health history may reveal, for example, that the occupant is afflicted by a communicable disease and the occupant's medical records may indicate symptoms of the communicable disease. The vehicle sanitizer system 108 may evaluate the information provided in the health history and may also, in some cases, confirm an identity and/or presence of the communicable disease by obtaining and evaluating information from one or more sensors in the vehicle 105. The vehicle sanitizer system 108 may then execute preventive and/or remedial actions (modifying air flow, instructing occupants, and/or carrying out disinfecting procedures) based on the health history of the occupant. In some cases, an occupant of the vehicle 105 may suffer from a disease that is not identifiable to the vehicle sanitizer system 108. In this situation, the vehicle sanitizer system 108 may informal all parties involved that additional research is needed to identify the disease and what actions should be taken and/or seek information from a medical authority (agency, hospital, research facility, doctor etc.), provide information to the medical authority, and/or alert the medical authority. All communications and storage or transmissions of data associated with health information will comply with all required regulations.

In certain embodiments, when the system determines a disease for the occupant from the health history of the occupant, but the system does not have corresponding information about symptoms associated with the disease, the system can use machine learning techniques to learn about the new disease for future identification/detection. For example, the system may know a number of diseases and symptoms associated with the diseases. If the occupant has a disease outside of the known diseases, the system may observe the occupant to learn about the disease. Also the system does not know what the symptoms are associated with the new disease since they are not pre-defined. In this manner, the system may observe the occupant (e.g., temperature above 100, etc.) to learn about the new disease. The occupant may or may not currently have the new disease or the occupant may have recovered from the new disease.

In an example embodiment, the vehicle sanitizer system 108 may wirelessly communicate with the personal communication device 111 for obtaining a travel history of the occupant of the vehicle 105. The travel history, which may be stored in a calendar, emails, or text in the personal communication device 111 may reveal, for example, that the occupant has recently traveled to a country afflicted by a communicable disease (influenza, for example) or an epidemic. The vehicle sanitizer system 108 may then execute preventive and/or remedial actions (modifying air flow, instructing occupants, and/or carrying out disinfecting procedures) based on the travel history.

In some cases, the travel history can pertain to a past usage of ride share services by an individual. The past usage can provide certain types of information that may be used by the vehicle sanitizer system 108 to identify a health status of the individual. A few examples of such information can include travel routes and travel times of the individual. The travel routes and/or travel times (a season of the year, for example) may be used by the vehicle sanitizer system 108 to obtain air quality information along the travel routes and execute sanitization procedures upon the vehicle 105 prior to the individual entering the vehicle 105.

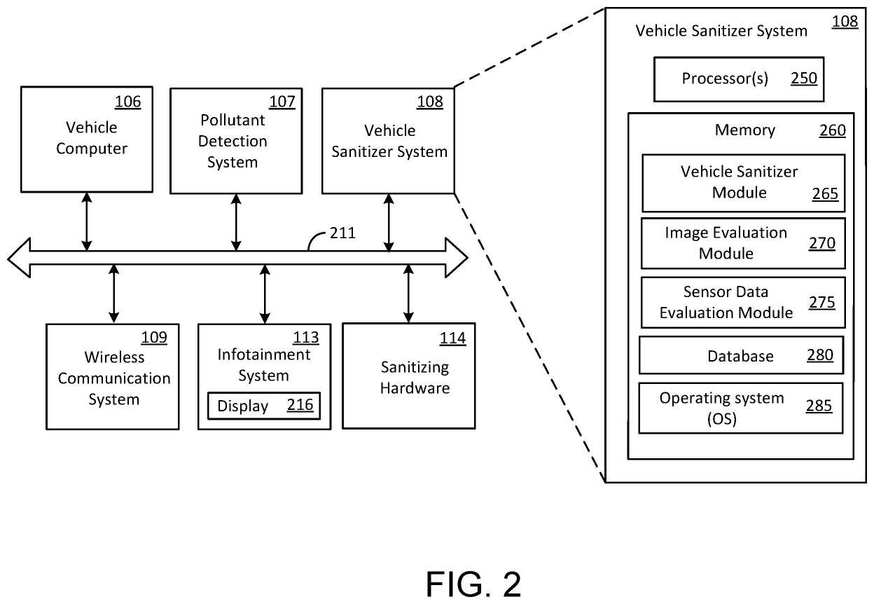

FIG. 2 shows some example components that may be included in the vehicle 105 in accordance with an embodiment of the disclosure. The example components can include the vehicle computer 106, the pollutant detection system 107, the vehicle sanitizer system 108, the wireless communication system 109, the infotainment system 113, and sanitizing hardware 114, which are communicatively coupled to each other via a bus 211.

The bus 211 can be implemented using one or more of various wired and/or wireless technologies. For example, the bus 211 can be a vehicle bus that uses a controller area network (CAN) bus protocol, a Media Oriented Systems Transport (MOST) bus protocol, and/or a CAN flexible data (CAN-FD) bus protocol. Some or all portions of the bus 211 may also be implemented using wireless technologies such as Bluetooth®, ZigBee®, or near-field-communications (NFC), cellular, Wi-Fi, Wi-Fi direct, machine-to-machine communication, and/or man-to-machine communication to accommodate communications between the vehicle sanitizer system 108 and various devices, such as, for example, the personal communication device 111 and devices coupled to the bus 211.

The bidirectional links between the various devices can carry commands in a first direction (such as, for example, a “fetch information” command issued by the vehicle sanitizer system 108 to the pollutant detection system 107 or to the sanitizing hardware 114) and/or can carry information in an opposite direction (such as, for example, images and/or sensor data from the pollutant detection system 107 to the vehicle sanitizer system 108).

The pollutant detection system 107 can include various types of components based on the nature of the detection process. For example, in one implementation, the pollutant detection system 107 may include a first camera that captures images of various objects in the cabin area of the vehicle 105 and a second camera that captures images of various objects outside the vehicle 105. One or both cameras may be a digital camera, a video camera, or an infrared imager. The images captured by the cameras may be propagated to the vehicle sanitizer system 108 for evaluation. In an example procedure, the vehicle sanitizer system 108 may evaluate a posture of an individual (slouched, stretched out, etc.) and/or a physical appearance (rashes, pallid, coughing, feverish, etc.) of the individual in the captured images and determine a health status of the individual.

In addition to the cameras, or in lieu of the cameras, the pollutant detection system 107 may include various types of sensors/detectors such as, for example, a thermal sensor, an infrared sensor, an audio sensor (a microphone, for example), photodiode sensors, and photodiode transmitters.

The wireless communication system 109 may include elements such as, for example, wireless transmitters and receivers that enable communicative coupling between the vehicle sanitizer system 108 and the network 130.

The infotainment system 113 can be an integrated unit that includes various components such as a radio, streaming audio solutions, and USB access ports for digital audio devices, with elements such as a navigation system that provides navigation instructions to the driver 112 of the vehicle 105. In an example implementation, the infotainment system 113 has a display 216 that includes a graphical user interface (GUI) for use by an occupant of the vehicle 105. The GUI may be used for various purposes such as to allow the driver 112 of the vehicle 105 to make a request to obtain navigation instructions and/or to sanitize the vehicle 105.

The display 216 may also be employed by the vehicle sanitizer system 108 to display various types of alerts and messages associated with sanitizing the vehicle 105. The vehicle sanitizer system 108, may, for example, instruct the driver 112 to exit the vehicle 105 so as to allow the sanitizing hardware 114 to sanitize the cabin area of the vehicle 105. The driver 112 may be further instructed to leave a window open when exiting the vehicle 105 so as to allow any disinfectant fumes that may be present in the cabin area of the vehicle 105 to be dispelled prior to re-use of the vehicle 105.

The GUI may be omitted in implementations where the vehicle 105 is an autonomous vehicle. In this scenario, the vehicle sanitizer system 108 may evaluate data and/or images received from the pollutant detection system 107 and make a determination that the cabin area is in need of sanitization. In one case, the autonomous vehicle may be a ride share vehicle and the need for sanitization may arise as a result of the pollutant detection system 107 detecting the presence of a virus in the cabin area after a ride share passenger has exited the vehicle 105 and the cabin area is unoccupied. The vehicle sanitizer system 108 may send a request to the sanitizing hardware 114 to sanitize the vehicle 105. The vehicle sanitizer system 108 may further communicate with the vehicle computer 106 to instruct the vehicle computer 106 to activate a window motor to open a window of the vehicle 105 when the sanitization is in progress.

The sanitizing hardware 114 may include various systems such as, for example, a dispensing system for dispensing sanitizing agents such as, for example, an antibacterial agent, a disinfectant, a sterilizer, an antiseptic, a deodorizer, or soap. In an example implementation, the sanitizing hardware 114 can include an ultraviolet (UV) light source arranged to shine UV light upon various surfaces and objects in the cabin area of the vehicle 105. The UV light source may be attached to a ceiling in the cabin area of the vehicle 105 so that the emitted UV light falls upon potentially contaminated object such as, for example, the seats, the dashboard, the windows, the floor, the interior panels, and the door handles.

The vehicle sanitizer system 108 may be provided in the form of a computer that includes a processor 250 and a memory 260. The memory 260, which is one example of a non-transitory computer-readable medium, may be used to store an operating system (OS) 285 and various code modules such as, for example, a vehicle sanitizer module 265, an image evaluation module 270, and a sensor data evaluation module 275. The code modules are provided in the form of computer-executable instructions that can be executed by the processor 250 for performing various operations in accordance with the disclosure.

The vehicle sanitizer module 265 may be executed by the processor 250 for performing various operations in accordance with the disclosure. These operations can include evaluating sensor data and/or camera images provided by the pollutant detection system 107. The sensor data may be evaluated in cooperation with the sensor data evaluation module 275 for executing various operations such as, for example, to determine a health risk that may be posed by a first occupant of the vehicle 105 to one or more other occupants of the vehicle 105. In the event of such a risk, the vehicle sanitizer module 265 may transmit a request to the vehicle computer 106 to modify an air quality in the cabin area of the vehicle 105.

The vehicle computer 106 may respond to the request in various ways. In an example implementation, the vehicle computer 106 may transmit a signal to a servomotor to adjust an air vent in a manner so as to direct airflow away from the first occupant and prevent contaminated air from reaching a second occupant of the vehicle 105.

In another example implementation, the vehicle computer 106 may cooperate with the sanitizing hardware 114 to dispense a disinfectant into the cabin area of the vehicle 105.

In another example implementation, the vehicle computer 106 may communicate with the infotainment system 113 to issue a notification to the occupants of the vehicle 105. The notification may be issued in the form of an audible warning through a speaker system of the vehicle 105 and/or in the form of a message that is displayed on the display 216 of the infotainment system 113. The audible warning and/or displayed message may alert the occupants to the health risk and may recommend actions to be taken to minimize exposure to the health risk. The recommended actions may include, for example, suggesting that every occupant don a mask, apply a sterilizer, use disinfectant wipes, and/or use a mouth covering (handkerchief, shirt sleeve, etc.) when coughing or sneezing.

In another example operation, the processor 250 may evaluate sensor data provided by an air quality sensor of the pollutant detection system 107 to the vehicle sanitizer system 108. The evaluation may include comparing a measured air quality parameter to a threshold air quality standard. If the measured air quality parameter is below the threshold air quality standard, the vehicle sanitizer system 108 may execute various actions to protect the occupants of the vehicle 105 such as, for example, shutting the air intake vent and re-circulating the air inside the cabin so as to prevent airborne pollutants (allergens, bacteria, etc.) from entering the vehicle 105.

In an example embodiment, a scheduling functionality may be included in the vehicle sanitizer module 265. In one implementation of this embodiment, the processor 250 may execute the scheduling functionality for performing a sanitizing operation based on a pre-defined schedule. In another implementation of this embodiment, the processor 250 may execute the scheduling functionality for performing an opportunistic sanitizing procedure. The opportunistic sanitizing procedure can involve automatically generating, updating, and/or modifying a sanitizing schedule to take advantage of lull periods where no occupants are present in the vehicle 105 (such as, for example, between trips in a ride share vehicle). In some cases, a customer who is scheduled for a ride in the vehicle 105 may be informed of a delay due to a sanitization procedure being carried out upon the vehicle 105. An alternative vehicle may be arranged to service the customer. In some cases, a passenger of the vehicle 105 may be informed that the vehicle 105 requires a sanitization procedure that requires the passenger to exit the vehicle 105. The sanitization procedure may be required as a result of an abnormal condition (for example, the passenger vomiting in the cabin area of the vehicle 105). In such a situation, an alternative arrangement may be made for the customer to continue on the ride. The alternative arrangement may, for example, involve dropping off the customer at an alternative location and/or arranging for a ride in another vehicle.

The database 280 may contain various types of information data than can be accessed by the processor 250 when evaluating sensor data and/or camera images to determine a health risk that may be posed by a first occupant of the vehicle 105 to a second occupant of the vehicle 105 (and other occupants). Such information can include, for example, symptoms associated with various diseases and remedial measures that can be taken to minimize risk of exposure to a communicable disease. In some cases, the database 280 may lack certain types of information such as, for example, real time data associated with a disease. In such cases, the processor 250 may obtain the information by communicating in real time via the network 130 with various information sources such as, for example, medical experts, hospitals, and news outlets. The processor 250 may also convey information to various entities for various purposes (such as, for example, to archive, to research, and/or to address issues).

In some implementations, the processor 250 may access and/or store sensor data in the database 280 for research purposes. In some instances, the sensor data may be processes via machine learning (or other artificial intelligence algorithms or the like) to better identify diseases and determine appropriate actions to take in future situations. In other instances, sensor data obtained by monitoring an occupant of the vehicle 105 may be transmitted to a research facility for studying a hitherto undiscovered or unevaluated disease.

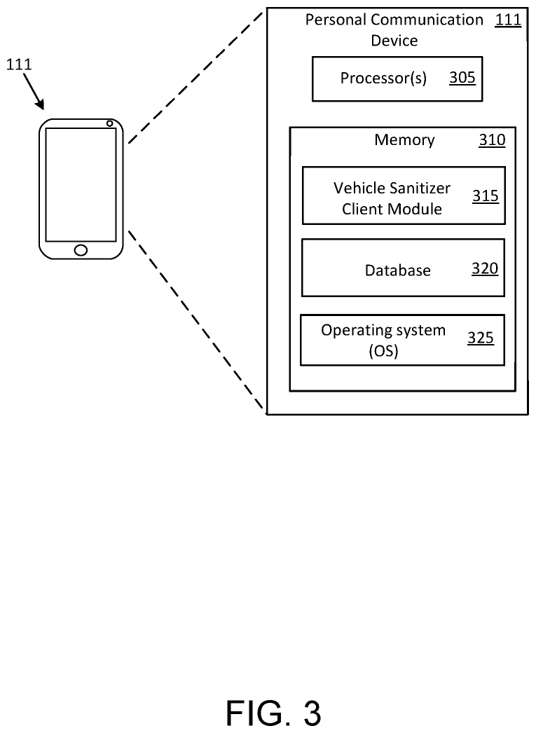

FIG. 3 shows some example components that may be included in the personal communication device 111 of an occupant of the vehicle 105 in accordance with an embodiment of the disclosure. The personal communication device 111 may include a processor 305 and a memory 310. The memory 310, which is yet another example of a non-transitory computer-readable medium, may be used to store an operating system (OS) 325, a database 320 and various code modules such as, for example, a vehicle sanitizer client module 315. The code modules are provided in the form of computer-executable instructions that can be executed by the processor 305 for performing various operations in accordance with the disclosure.

The vehicle sanitizer client module 315, which may be downloaded into the personal communication device 111 in the form of a software application, may be executed by the processor 305 for performing various operations in accordance with the disclosure. In an example operation, the processor 305 may communicate with the vehicle sanitizer system 108 when determining a health risk that may be posed by the occupant who owns the personal communication device 111 to other occupants of the vehicle 105, and to display messages on a display screen of the personal communication device 111.

As a part of the evaluation procedure, the processor 305 of the personal communication device 111 may access the database 320 to obtain various types of information such as for example, a health history and/or a travel history of the occupant of the vehicle 105. While some of such information may be stored in the database 320, other information may be available to the processor 305 from other information sources such as for example, a doctor's office. In an example scenario, the processor 305 may access email, text, rider profile and a calendar in the personal communication device 111 for obtaining travel history and/or upcoming travel information of the occupant of the vehicle 105. The travel history may reveal, for example, that the occupant has recently traveled to a country afflicted by a communicable disease (influenza, for example) or an epidemic.

The health history of the occupant of the vehicle 105 may not only provide particulars of a health issue that the occupant may have, but also information about medical personnel and medical institutions that may provide additional information about the health of the occupant of the vehicle 105.

In an example embodiment, the vehicle sanitizer client module 315 in the personal communication device 111 and/or the vehicle sanitizer module 265 of the vehicle sanitization system 108, may be configured to operate as a digital health companion. Accordingly, when executed by the processor 305, the vehicle sanitizer client module 315 may provide guidance to the occupant of the vehicle 105 with respect to health issues such as, for example, educating the occupant on measures that may be taken to protect against a communicable disease.

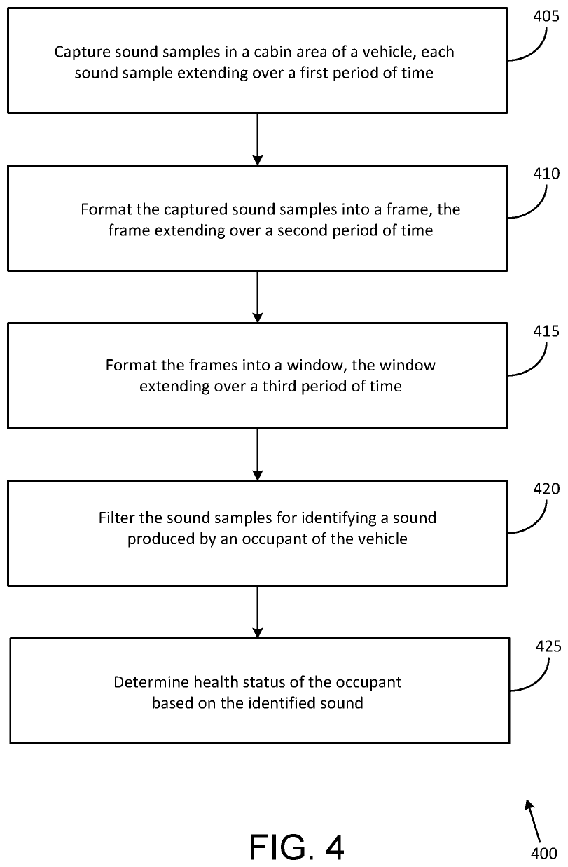

FIG. 4 shows a flowchart 400 of an example procedure to analyze a sound produced by an occupant of the vehicle 105 in order to identify a health status of the occupant. The flowchart 400 illustrates a sequence of operations that can be implemented in hardware, software, or a combination thereof. In the context of software, the operations represent computer-executable instructions stored on one or more non-transitory computer-readable media such as the memory 260, that, when executed by one or more processors such as the processor 250, perform the recited operations. Generally, computer-executable instructions include routines, programs, objects, components, data structures, and the like that perform particular functions or implement particular abstract data types. The order in which the operations are described is not intended to be construed as a limitation, and any number of the described operations may be carried out in a different order, omitted, combined in any order, and/or carried out in parallel.

Some or all of the operations described in the flowchart 400 may be carried out by the vehicle sanitizer system 108, the pollutant detection system 107, the vehicle computer 106, and/or the sanitizing hardware 114 provided in the vehicle 105. The description below makes reference to certain components and objects shown in FIGS. 1-3, but it should be understood that this is done primarily for purposes of describing certain aspects of the disclosure and that the description is equally applicable to various other embodiments.

At block 405, sound samples may be captured in a cabin area of the vehicle 105. The sound samples may be captured by an audio sensor (a microphone, for example) located in the cabin area, and propagated to the vehicle sanitizer system 108. The vehicle sanitizer system 108 may evaluate the sound samples to determine whether the sound corresponds to, a cough, a sneeze, or a wheeze, for example. It must be understood that a cough, a sneeze, or a wheeze are merely a few examples of sounds that may be symptomatic of a health issue associated with an occupant of the vehicle 105 (fever, cold, flu, viral infection, etc.).

Each sound sample may be captured over a first period of time that may be selected in correspondence to a typical length of time a person may cough, sneeze, or wheeze. In an example implementation, the first period of time may range from about 2 seconds to about a minute. The first period of time and other time periods referred to herein are merely examples, and other time periods may be used in other applications. Such time periods may be selected in accordance with the nature of the sound to be evaluated. For example, a time period associated with a sneeze may be typically smaller than a time period associated with a hacking cough.

At block 410, the captured sound samples may be arranged in a frame format. The frame format may extend over a second period of time that may be selected to encompass “n” number of sound samples. In an example implementation, the second period of time may range from about 10 seconds to about 5 minutes so as to accommodate five sound samples (n=5) captured over the example first period of time referred to above (about 2 seconds to about a minute).

At block 415, the frames may be arranged in a window format. The window format may extend over a third period of time that may be selected to encompass “m” frames. In an example implementation, the third period of time may range from about 1 minute to about 30 minutes so as to accommodate six frames corresponding to the example second period of time referred to above (about 10 seconds to about 5 minutes).

At block 420, the captured sound samples that have been formatted in the manner described above may be filtered so as eliminate irrelevant ambient sounds and isolate unique sound patterns that are characteristically associated with sounds such as a cough, a sneeze, or a wheeze, for example. Irrelevant ambient sounds may include, for example, human speech, music, and vehicle-related sounds (engine, air conditioner, etc.). The filtered sound may be classified and analyzed for determining a nature of the sound (cough, sneeze, wheeze, etc.). In an example embodiment, the filtering may be carried out by applying the following equation:

$$R M S ( f ) = \frac{{Σ}_{i = 1}^{n} ( S {i}^{2} )}{n}$$

where “f” corresponds to a frame, Si corresponds to a sample in a frame, and “n” corresponds to a total number of samples in a frame.

At block 425, a health status of the occupant of the vehicle 105 may be determined based on the nature of the sound identified after filtering.

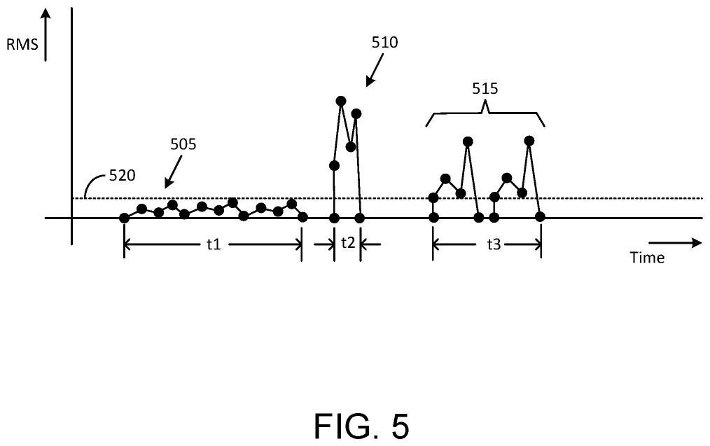

FIG. 5 shows a graphical representation of various types of example sound patterns that may be analyzed in order to determine a health status of an occupant of the vehicle 105. The amplitude and time characteristics of a first example sound pattern 505 may be attributable to the occupant talking with another occupant, or talking into the personal communication device 111. An RMS average amplitude of the first example sound pattern 505 is indicated by an amplitude level 520. The first example sound pattern 505 may extend over a time period “t1” (several minutes, for example).

The amplitude and time characteristics of a second example sound pattern 510 may be attributable to a sneeze, which is generally characterized by a sharp rise in amplitude over a short period of time. An RMS average amplitude of the second example sound pattern 510 exceeds the amplitude level 520. The second example sound pattern 510 may extend over a time period “t2” that is less than the time period “t1.”

The amplitude and time characteristics of a third example sound pattern 515 may be attributable to a coughing fit, which is generally characterized by repetitive sound bursts over a period time, each sound burst having a sharp rise in amplitude over a short period of time. An RMS average amplitude of the third example sound pattern 515 also exceeds the amplitude level 520. The third example sound pattern 515 may extend over a time period “t3” that is less than the time period “t1” and greater than the time period “t2.”

Various sound patterns such as the example sound patterns describe above, may be analyzed in various ways to identify the nature of the sound. In a first example implementation, a statistics-based approach may be used. In a second example implementation, one or more templates may be used. In a third example implementation, a human may evaluate and analyze the sound.

Furthermore, after identifying the nature of the sound the vehicle sanitizer system 108 may use information on travel history and health status of the occupant to determine a level of risk (low risk, medium risk, or high risk, for example). For example, occasional throat clearing or sniffing, normal body temperature, and no travel history to epidemic areas may be classified as low risk. Stray cough, stray sneeze, and mild fever may be classified as medium risk. Sustained bouts of coughing, sustained sneezing, and travel to epidemic areas may be classified as high risk. The vehicle sanitizing system 108 may use the risk level to determine various actions to be performed during sanitization. For example, the vehicle sanitizing system 108 may perform a disinfection procedure at a previously scheduled time when the risk level is low and may perform a disinfection procedure immediately or at an accelerated schedule when the risk level is high.

In the above disclosure, reference has been made to the accompanying drawings, which form a part hereof, which illustrate specific implementations in which the present disclosure may be practiced. It is understood that other implementations may be utilized, and structural changes may be made without departing from the scope of the present disclosure. References in the specification to “one embodiment,” “an embodiment,” “an example embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, one skilled in the art will recognize such feature, structure, or characteristic in connection with other embodiments whether or not explicitly described.

Implementations of the systems, apparatuses, devices, and methods disclosed herein may comprise or utilize one or more devices that include hardware, such as, for example, one or more processors and system memory, as discussed herein. An implementation of the devices, systems, and methods disclosed herein may communicate over a computer network. A “network” is defined as one or more data links that enable the transport of electronic data between computer systems and/or modules and/or other electronic devices. When information is transferred or provided over a network or another communications connection (either hardwired, wireless, or any combination of hardwired or wireless) to a computer, the computer properly views the connection as a transmission medium. Transmission media can include a network and/or data links, which can be used to carry desired program code means in the form of computer-executable instructions or data structures and which can be accessed by a general purpose or special purpose computer. Combinations of the above should also be included within the scope of non-transitory computer-readable media.

Computer-executable instructions comprise, for example, instructions and data which, when executed at a processor, such as the processor 250 or the processor 305, cause the processor to perform a certain function or group of functions. The computer-executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

A memory device such as the memory 260 or the memory 310, can include any one memory element or a combination of volatile memory elements (e.g., random access memory (RAM, such as DRAM, SRAM, SDRAM, etc.)) and non-volatile memory elements (e.g., ROM, hard drive, tape, CDROM, etc.). Moreover, the memory device may incorporate electronic, electromagnetic, optical, and/or other types of storage media. In the context of this document, a “non-transitory computer-readable medium” can be, for example but not limited to, an electronic, electromagnetic, optical, electromagnetic, infrared, or semiconductor system, apparatus, or device. More specific examples (a non-exhaustive list) of the computer-readable medium would include the following: a portable computer diskette (electromagnetic), a random-access memory (RAM) (electronic), a read-only memory (ROM) (electronic), an erasable programmable read-only memory (EPROM, EEPROM, or Flash memory) (electronic), and a portable compact disc read-only memory (CD ROM) (optical). Note that the computer-readable medium could even be paper or another suitable medium upon which the program is printed, since the program can be electronically captured, for instance, via optical scanning of the paper or other medium, then compiled, interpreted or otherwise processed in a suitable manner if necessary, and then stored in a computer memory.

Those skilled in the art will appreciate that the present disclosure may be practiced in network computing environments with many types of computer system configurations, including in-dash vehicle computers, personal computers, desktop computers, laptop computers, message processors, handheld devices, multi-processor systems, microprocessor-based or programmable consumer electronics, network PCs, minicomputers, mainframe computers, mobile telephones, PDAs, tablets, pagers, routers, switches, various storage devices, and the like. The disclosure may also be practiced in distributed system environments where local and remote computer systems, which are linked (either by hardwired data links, wireless data links, or by any combination of hardwired and wireless data links) through a network, both perform tasks. In a distributed system environment, program modules may be located in both the local and remote memory storage devices.

Further, where appropriate, the functions described herein can be performed in one or more of hardware, software, firmware, digital components, or analog components. For example, one or more application specific integrated circuits (ASICs) can be programmed to carry out one or more of the systems and procedures described herein. Certain terms are used throughout the description, and claims refer to particular system components. As one skilled in the art will appreciate, components may be referred to by different names. This document does not intend to distinguish between components that differ in name, but not in function.

It should be noted that the sensor embodiments discussed above may comprise computer hardware, software, firmware, or any combination thereof to perform at least a portion of their functions. For example, a sensor may include computer code configured to be executed in one or more processors and may include hardware logic/electrical circuitry controlled by the computer code. These example devices are provided herein for purposes of illustration and are not intended to be limiting. Embodiments of the present disclosure may be implemented in further types of devices, as would be known to persons skilled in the relevant art(s).

At least some embodiments of the present disclosure have been directed to computer program products comprising such logic (e.g., in the form of software) stored on any computer-usable medium. Such software, when executed in one or more data processing devices, causes a device to operate as described herein.

While various embodiments of the present disclosure have been described above, it should be understood that they have been presented by way of example only, and not limitation. It will be apparent to persons skilled in the relevant art that various changes in form and detail can be made therein without departing from the spirit and scope of the present disclosure. Thus, the breadth and scope of the present disclosure should not be limited by any of the above-described example embodiments but should be defined only in accordance with the following claims and their equivalents. The foregoing description has been presented for the purposes of illustration and description. It is not intended to be exhaustive or to limit the present disclosure to the precise form disclosed. Many modifications and variations are possible in light of the above teaching. Further, it should be noted that any or all of the aforementioned alternate implementations may be used in any combination desired to form additional hybrid implementations of the present disclosure. For example, any of the functionality described with respect to a particular device or component may be performed by another device or component. Further, while specific device characteristics have been described, embodiments of the disclosure may relate to numerous other device characteristics. Further, although embodiments have been described in language specific to structural features and/or methodological acts, it is to be understood that the disclosure is not necessarily limited to the specific features or acts described. Rather, the specific features and acts are disclosed as illustrative forms of implementing the embodiments. Conditional language, such as, among others, “can,” “could,” “might,” or “may,” unless specifically stated otherwise, or otherwise understood within the context as used, is generally intended to convey that certain embodiments could include, while other embodiments may not include, certain features, elements, and/or steps. Thus, such conditional language is not generally intended to imply that features, elements, and/or steps are in any way required for one or more embodiments.

That which is claimed is:

1. A method comprising:
obtaining a health history and/or a travel history of a first occupant of a vehicle;
evaluating the health history and/or the travel history of the first occupant to determine a first health risk posed by the first occupant to a second occupant of the vehicle; and
modifying an air quality inside the vehicle based on the first health risk posed by the first occupant to the second occupant of the vehicle.

2. The method of claim 1, wherein the travel history of the first occupant comprises travel to a country having an epidemic.

3. The method of claim 1, wherein the health history of the first occupant includes an indication that the first occupant suffers from a communicable disease.

4. The method of claim 1, further comprising:
detecting, by a sensor, a symptomatic feature that indicates a health status of the first occupant;
producing, by the sensor, sensor data based on detecting the symptomatic feature;
propagating, by the sensor, the sensor data;
evaluating the sensor data; and
identifying, based on evaluating the sensor data, that the first occupant poses a second health risk to the second occupant.

5. The method of claim 4, wherein the symptomatic feature that indicates the health status of the first occupant is a body temperature of the first occupant, and wherein evaluating the sensor data comprises comparing the body temperature of the first occupant to a threshold body temperature.

6. The method of claim 4, wherein the symptomatic feature that indicates the health status of the first occupant is a sound emitted by the first occupant, and wherein evaluating the sensor data comprises analyzing the sound to detect a cough, a sneeze, and/or a wheeze.

7. The method of claim 6, wherein analyzing the sound to detect the cough, the sneeze, and/or the wheeze comprises executing a pattern recognition algorithm.

8. A method comprising:
detecting, by a first sensor, a symptomatic feature that indicates a health status of a first occupant of a vehicle;
producing, by the first sensor, first sensor data based on detecting the symptomatic feature;
propagating, by the first sensor, to a computer, the first sensor data;
evaluating, by the computer, the first sensor data; and
identifying, by the computer, based on evaluating the first sensor data, a first health risk posed by the first occupant to a second occupant of the vehicle.

9. The method of claim 8, further comprising:
modifying, by the computer, an air quality inside the vehicle based on the first health risk posed by the first occupant to the second occupant of the vehicle.

10. The method of claim 8, wherein the symptomatic feature that indicates the health status of the first occupant is a body temperature of the first occupant, and wherein evaluating the first sensor data comprises comparing the body temperature of the first occupant to a threshold body temperature.

11. The method of claim 8, wherein the symptomatic feature that indicates the health status of the first occupant is a sound emitted by the first occupant, and wherein evaluating the first sensor data comprises analyzing the sound to detect a cough, a sneeze, and/or a wheeze.

12. The method of claim 11, wherein analyzing the sound to detect the cough, the sneeze, and/or the wheeze comprises executing a pattern recognition algorithm and the method further comprises:
issuing an instruction to the first occupant and/or the second occupant to don a mask.

13. The method of claim 8, further comprising:
detecting, by a second sensor, a first air quality outside the vehicle;
producing, by the second sensor, second sensor data based on detecting the first air quality outside the vehicle;
propagating, by the second sensor, to the computer, the second sensor data;
evaluating, by the computer, the first sensor data to determine a second health risk posed by the first air quality to the first occupant and/or the second occupant; and
modifying, by the computer, a second air quality inside the vehicle based on the first health risk and/or the second health risk.

14. The method of claim 13, wherein detecting the first air quality outside the vehicle comprises detecting a pollutant level in air outside the vehicle.

15. A vehicle comprising:
a first sensor; and
a memory that stores computer-executable instructions; and
a processor configured to access the memory and execute the computer-executable instructions to perform operations comprising:
receiving, from the first sensor, first sensor data based on detecting a symptomatic feature that indicates a health status of a first occupant of the vehicle;
evaluating the first sensor data; and
identifying, based on evaluating the first sensor data, a first health risk posed by the first occupant to a second occupant of the vehicle.

16. The vehicle of claim 15, wherein the first sensor is one of a thermal sensor or an infrared imager, wherein the symptomatic feature that indicates the health status of the first occupant is a body temperature of the first occupant, and wherein evaluating the first sensor data comprises comparing the body temperature of the first occupant to a threshold body temperature.

17. The vehicle of claim 15, wherein the first sensor is an audio sensor, wherein the symptomatic feature that indicates the health status of the first occupant is a sound emitted by the first occupant, and wherein evaluating the first sensor data comprises analyzing the sound to detect a cough, a sneeze, and/or a wheeze.

18. The vehicle of claim 17, wherein analyzing the sound to detect the cough, the sneeze, or the wheeze comprises executing a pattern recognition algorithm.

19. The vehicle of claim 15, further comprising a second sensor, and wherein the processor is further configured to access the memory and execute additional computer-executable instructions to perform operations comprising:
receiving, from the second sensor, second sensor data based on detecting a first air quality outside the vehicle;
evaluating the second sensor data to determine a second health risk posed by the first air quality to the first occupant and/or the second occupant; and
modifying a second air quality inside the vehicle based on the first health risk and/or the second health risk.

20. The vehicle of claim 19, wherein the second sensor is an air quality sensor, and wherein detecting the first air quality outside the vehicle comprises detecting a pollutant level in air outside the vehicle.
